(19) United States
(12) Patent Application Publication
(10) Pub. No.: US 2016/0245786 A1
(43) Pub. Date: Aug. 25, 2016

(54) LIPID BIOMARKERS OF HEALTHY AGEING

(71) Applicant: NESTEC S.A., Vevey (CH)

(72) Inventors: Sebastiano Collino, Lausanne (CH); Ivan Montoliu Roura, Lausanne 26 (CH); Françios-Pierre Martin, Vuisternens-devant-Romont (CH); Fiona Camille Beguelin, Lausanne (CH); Serge André Dominique Rezzi, Semsales (CH)

(21) Appl. No.: 15/028,244

(22) PCT Filed: Nov. 5, 2014
(86) PCT No.: PCT/EP2014/073781

§ 371 Date: Apr. 8, 2016

(30) Foreign Application Priority Data
Nov. 14, 2013 (EP) 13192937.4

(51) Int. Cl. G01N 33/487

(52) U.S. Cl. CPC G01N 33/487; G01N 2405/00; G01N 2800/50; G01N 2560/00; G01N 2405/02; G01N 2405/04; G01N 2405/08; G01N 2570/00

(57) ABSTRACT

In one aspect there is provided a method for predicting a risk of unhealthy ageing in a subject, comprising: (a) determining a level of two or more lipid biomarkers in a sample from the subject, wherein the biomarkers are selected from two or more of the following groups: (i) a triacylglycerol (TAG) from TAG (46:5) to TAG (54:3); (ii) an ether phosphatidylcholine (PC-O) from PC-O(28:0) to PC-O(38:6); (iii) a sphingomyelin (SM) from SM (33:1) to SM(50:1); (iv) a phosphatidylcholine (PC) from PC (32:1) to PC (40:5); (v) a phosphatidylinositol (PI) from PI (36:1) to PI (38:3); (vi) a phosphatidylethanolamine (PE) from PE (36:2) to PE (38:4); and (b) comparing the levels of the biomarkers in the sample to reference values; wherein the levels of the biomarkers in the sample compared to the reference values are indicative of the risk of unhealthy ageing in the subject.

### FIELD OF THE INVENTION

The present invention generally concerns a healthy lifestyle and the prevention of age-related chronic disorders. In particular, the present invention concerns biomarkers and their use to monitor the ageing process. As such, the present invention provides a number of lipid biomarkers and biomarker combinations that can be used to predict a risk of unhealthy ageing in a subject.

### BACKGROUND

Aging is defined as the time-dependent decline of functional capacity and stress resistance, associated with increased risk of morbidity and mortality. Additionally, the aging phenotype in humans is very heterogeneous and can be described as a complex mosaic resulting from the interaction of a variety of environmental, stochastic and genetic-epigenetic variables. Decades of research on aging have found hundreds of genes and many biological processes that are associated to the aging process, but at the same time, many fundamental questions are still unanswered or are the object of intense debate.

These questions are frequently not addressable by examining a single gene or a single pathway, but are better addressed at a systemic level, capturing aging as a complex multi-factorial process. Moreover, ageing is accompanied by a chronic, low grade, inflammatory status, resulting from an imbalance between pro- and anti-inflammatory processes, a pathogenic condition that has been revealed critical in the onset of major age-related chronic diseases such as atherosclerosis, type 2 diabetes, and neurodegeneration.

Within this perspective, acquired healthy aging and longevity are likely the reflection of not only a lower propensity to accumulate inflammatory responses, but also of efficient anti-inflammatory network development. In addition, there is a growing awareness of the importance of the variation in the gut microbiota as its effects on the host mammalian system, having displayed direct influence in the etiology of several diseases such as insulin resistance, Crohn's disease, irritable bowel syndrome, obesity, and cardiovascular disease.

Metabonomics is considered today a well-established system approach to characterize the metabolic phenotype, which results from a coordinated physiological response to various intrinsic and extrinsic parameters including environment, drugs, dietary patterns, lifestyle, genetics, and microbiome. Unlike gene expression and proteomic data which indicate the potential for physiological changes, metabolites and their kinetic changes in concentration within cells, tissues and organs, represent the real end-points of physiological regulatory processes.

Metabolomics had successfully been applied to study the modulation of the ageing processes following nutritional interventions, including caloric restriction-induced metabolic changes in mice, dogs, and non-human primates. Specifically, in the canine population profound changes in gut microbiota metabolism were associated with ageing. Despite these findings, a comprehensive profiling of the molecular mechanisms affecting the aging process has not yet been reported. Moreover, metabolic phenotyping of longevity is still missing.

In order to better elucidate molecular mechanisms that involve the disruption of lipid metabolic pathways, the field of lipidomics is being used. Lipidomics can be performed by a comprehensive measurement of the lipidome, i.e. the complete set of biological lipids, from a single analysis in a non-targeted profiling way (shotgun approach). However, there is still a need for the identification of reliable lipid biomarkers which are indicative of healthy and unhealthy ageing in a subject.

Consequently, it was the objective of the present invention to provide lipid biomarkers that can be detected easily and that facilitate the prediction of the risk of healthy or unhealthy ageing in a subject. Such lipid biomarkers can be used to promote healthy ageing by identifying subjects at increased risk of unhealthy ageing, and modifying the lifestyle of such subjects accordingly. This may permit the delay of ageing related chronic inflammatory disorders in the subjects.

### SUMMARY OF THE INVENTION

Accordingly the present invention provides in one aspect a method for predicting a risk of unhealthy ageing in a subject, comprising: (a) determining a level of two or more lipid biomarkers in a sample from the subject, wherein the biomarkers are selected from two or more of the following groups: (i) a triacylglycerol (TAG) from TAG(46:1) to TAG(54:6); (ii) an ether phosphatidylcholine (PC-O) from PC-O(28:0) to PC-O(38:6); (iii) a sphingomyelin (SM) from SM(33:1) to SM(50:4); (iv) a phosphatidylcholine (PC) from PC(32:1) to PC(40:5); (v) a phosphatidylinositol (PI) from PI(36:1) to PI(38:3); (vi) a phosphatidylethanolamine (PE) from PE(36:2) to PE(38:4); and (b) comparing the levels of the biomarkers in the sample to reference values; wherein the levels of the biomarkers in the sample compared to the reference values are indicative of the risk of unhealthy ageing in the subject.

In one embodiment the method comprises determining a level of: (i) a triacylglycerol (TAG) from TAG(46:1) to TAG(54:6); and (ii) an ether phosphatidylcholine (PC-O) from PC-O(28:0) to PC-O(38:6) in the sample from the subject.

In the embodiment of the preceding paragraph, the method preferably further comprises determining a level of a sphingomyelin (SM) from SM(33:1) to SM(50:4) in the sample from the subject.

In the embodiment of either of the two preceding paragraphs, the method preferably further comprises determining a level of a phosphatidylethanolamine (PE) from PE(36:2) to PE(38:4) in the sample from the subject.

In the embodiment of any of the three preceding paragraphs, the method preferably further comprises determining a level of a phosphatidylinositol (PI) from PI(36:1) to PI(38:3) in the sample from the subject.

In the embodiment of any of the four preceding paragraphs, the method preferably further comprises determining a level of a phosphatidylcholine (PC) from PC(32:1) to PC(40:5) in the sample from the subject.

In one embodiment, a level of a TAG from TAG(46:5) to TAG(47:5) is determined, and an increase in the level of the TAG in the sample from the subject compared to the reference value is indicative of an increased risk of unhealthy ageing in the subject. Preferably the TAG is TAG(46:5) or TAG(47:5).

In another embodiment, a level of a TAG from TAG(48:1) to TAG(54:6) is determined, and a decrease in the level of the TAG in the sample from the subject compared to the reference value is indicative of an increased risk of unhealthy ageing in the subject. Preferably the TAG is TAG(48:6), TAG(52:2) or TAG(54:3).

In one embodiment, a level of a PC-O from PC-O(28:0) to PC-O(30:0) is determined, and an increase in the level of the PC-O in the sample from the subject compared to the reference value is indicative of an increased risk of unhealthy ageing in the subject. Preferably the PC-O is PC-O(28:0) or PC-O(30:0).

In another embodiment, a level of a PC-O from PC-O(32:1) to PC-O(38:6) is determined, and a decrease in the level of the PC-O in the sample from the subject compared to the reference value is indicative of an increased risk of unhealthy ageing in the subject. Preferably the PC-O is PC-O(32:1), PC-O(34:1), PC-O(34:2), PC-O(36:3), PC-O(38:4), PC-O(38:5) or PC-O(38:6).

In one embodiment, a level of a SM from SM(33:1) to SM(42:4) is determined, and a decrease in the level of the SM in the sample from the subject compared to the reference value is indicative of an increased risk of unhealthy ageing in the subject. Preferably the SM is SM(33:1), SM(34:1), SM(36:1), SM(36:2), SM(38:2), SM(41:2), SM(42:2), SM(42:3) or SM(42:4).

In another embodiment, a level of SM(50:1) is determined, and an increase in the level of the SM in the sample from the subject compared to the reference value is indicative of an increased risk of unhealthy ageing in the subject.

In one embodiment, a level of a PE from PE(36:2) to PE(38:4) is determined, and a decrease in the level of the PE in the sample from the subject compared to the reference value is indicative of an increased risk of unhealthy ageing in the subject.

In one embodiment, a level of a PI from PI(36:1) to PI(38:3) is determined, and a decrease in the level of the PI in the sample from the subject compared to the reference value is indicative of an increased risk of unhealthy ageing in the subject. Preferably the PI is PI(18:1-16:0) or PI(20:3-18:0).

In one embodiment, a level of a PC from PC(32:1) to PC(40:5) is determined, and a decrease in the level of the PC in the sample from the subject compared to the reference value is indicative of an increased risk of unhealthy ageing in the subject. Preferably the PC is PC(14:0-18:1) or PC(16:0-18:1).

In one embodiment, the sample comprises serum or plasma obtained from the subject.

In one embodiment, the reference value is based on a mean level of the biomarker in a control population of subjects.

In one embodiment, the levels of the biomarkers are determined by mass spectrometry.

In one embodiment, the levels of the biomarkers in the sample compared to the reference values are indicative of the risk of developing chronic age-related inflammatory disease in the subject.

In another embodiment, the levels of the biomarkers in the sample compared to the reference values are indicative of longevity of the subject.

In a further aspect, the present invention provides a method for promoting healthy ageing in a subject, comprising: (a) performing a method for predicting a risk of unhealthy ageing as described above; and (b) modifying a lifestyle of the subject if the subject has levels of the biomarkers which are indicative of an increased risk of unhealthy ageing.

In one embodiment, the modification in lifestyle in the subject comprises a change in diet. Preferably the change in diet comprises administering at least one nutritional product to the subject that reduces the risk of the development of chronic age-related inflammatory disease in the subject.

For example, the change in diet could, but is not limited to, reduction of carbohydrates, reduction of fats, weight control, reduction of alcohol consumption, increasing physical activity and maintaining a low-fat or very-low-fat diet.

In a preferred embodiment the change in diet comprises administering at least one nutritional product to a subject which is reduces the risk of development of chronic age-related inflammatory disease in the subject.

Examples of nutritional interventions include, but are not limited to, omega-3 fatty acid (e.g., fish oil), phytosterols, long chain polyunsaturated fatty acids (LC-PUFA), taurine, probiotics, carbohydrates, proteins, dietary fiber, phytonutrients, and combinations thereof.

For example, the change in diet may comprise increased consumption of fish, fish oil, omega-3 polyunsaturated fatty acids, zinc, vitamin E and/or B vitamins.

In some embodiments, the nutritional intervention comprises a food product including milk-powder based products, instant drinks, ready-to-drink formulations, nutritional powders; milk-based products (e.g., yogurt or ice cream), cereal products, beverages, water, teas (e.g., green tea or oolong tea), coffee, espresso based drinks, malt drinks, chocolate flavored drinks, culinary products and soups.

In another embodiment, the nutritional agent is a nutritionally complete formula.

In one embodiment, the method comprises a further step of repeating the method for predicting a risk of unhealthy ageing in the subject, after modifying the lifestyle of the subject.

In a further aspect, the present invention provides a method for predicting a risk of unhealthy ageing in a subject, comprising:

(a) determining a level of a triacylglycerol (TAG) from TAG(46:1) to TAG(54:6) in a sample from the subject; and

(b) comparing the levels of the TAG in the sample to a reference value;

wherein the levels of the TAG in the sample compared to the reference value is indicative of the risk of unhealthy ageing in the subject.

### DETAILED DESCRIPTION OF THE INVENTION

### Predicting a Risk of Unhealthy Ageing in a Subject

The present invention relates in one aspect to a method of predicting a risk of unhealthy ageing in a subject. In particular embodiments, the method may be used to diagnose unhealthy ageing, to monitor the progression of unhealthy ageing or to identify subjects at risk of unhealthy ageing. For instance, the method may be used to predict the likelihood of the subject ageing in an unhealthy manner, or to assess the current extent of unhealthy ageing in the subject. The method may also be used to assess the efficacy of an intervention to promote healthy ageing, for instance to monitor the effectiveness of a lifestyle change or change in diet in promoting healthy ageing.

The method may also be used to diagnose healthy ageing, for instance to predict a likelihood of healthy ageing in a subject or to identify subjects likely to age healthily.

In some embodiments, the method may be used to predict a risk of developing chronic age-related inflammatory disease in the subject. In other embodiments, the method may be used to predict longevity of the subject. Typical age-related chronic inflammatory disorders are known to those of skill in the art. A large part of the ageing phenotype is explained by an imbalance between inflammatory and anti-inflammatory networks, which results in the low grade chronic pro-inflammatory status of ageing, “inflamm-ageing” (Candore G., et al., Biogerontology. 2010 October; 11(5):565-73).

Typical age related inflammatory disorders include atherosclerosis, arthritis, dementia, type 2 diabetes, osteoporosis, and cardiovascular diseases, for example. For example for these disorders inflammation is seen as a possible underlying basis for the molecular alterations that link aging and age related pathological processes (Chung et al., ANTIOXIDANTS & REDOX SIGNALING, Volume 8, Numbers 3 & 4, 2006, 572-581).

### Subject

The present method may be carried out on any subject, including non-human or human subjects. In one embodiment, the subject is a mammal, preferably a human. The subject may alternatively be a non-human mammal, including for example a horse, cow, sheep or pig. In one embodiment, the subject is a companion animal such as a dog or cat.

The subject may be of any age, but is preferably a middle-aged or an elderly subject. For instance the subject may be in the age range 40 to 100 years, more preferably 40 to 80 years. The subject may be of either sex. However in one embodiment, the subject is female.

### Sample

The present method comprises a step of determining the level of two or more lipid biomarkers in a sample obtained from a subject. Thus the present method is typically practiced outside of the human or animal body, e.g. on a body fluid sample that was previously obtained from the subject to be tested. Preferably the sample is derived from blood, i.e. the sample comprises whole blood or a blood fraction. Most preferably the sample comprises blood plasma or serum.

Techniques for collecting blood samples and separating blood fractions are well known in the art. For instance, vena blood samples can be collected from patients using a needle and deposited into plastic tubes. The collection tubes may, for example, contain spray-coated silica and a polymer gel for serum separation. Serum can be separated by centrifugation at 1300 RCF for 10 min at room temperature and stored in small plastic tubes at −80° C.

### Determining Levels of Lipid Biomarkers in the Sample

The levels of individual lipid species in the sample may be measured or determined by any suitable method. For example, nuclear magnetic resonance spectroscopy (¹H-NMR) or mass spectroscopy (MS) may be used. Other spectroscopic methods, chromatographic methods, labeling techniques, or quantitative chemical methods may be used in alternative embodiments. Most preferably, the lipid levels in the sample are measured by mass spectroscopy. Typically the lipid level in the sample and the reference value are determined using the same analytical method.

### Lipids

The present method involves determining the levels of two or more lipid biomarkers selected from triacylglycerols (TAGs), ether phosphatidylcholines (PC-Os), sphingomyelins (SMs), phosphatidylcholines (PCs), phosphatidylinositols (PIs) and phosphatidylethanolamines (PEs). Typically the method involves measuring levels of at least one biomarker from each of two or more of the above groups. By combining measurements of biomarkers from multiple lipid groups, the present invention provides an improved lipid biomarker signature of healthy ageing, which can be used to identify subjects requiring intervention to prevent the development of age-related conditions.

### Triacylglycerols

In one embodiment, a triacylglycerol (TAG) from TAG(46:1) to TAG(54:6) is determined. In the nomenclature (X:Y), X refers to the total number of carbon atoms in the fatty acid portions of the molecule, and Y defines the total number of double bonds in the fatty acid portions of the molecule. Thus a triacylglycerol (TAG) from TAG(46:1) to TAG(54:6) refers to a TAG comprising 46 to 54 carbon atoms in the fatty acid chains and 1 to 6 double bonds in the fatty acid chains. The present method may involve determining the level of one or more such individual species of TAG.

In a preferred embodiment, the TAG comprises 46 or 47 carbon atoms in the fatty acid chains. In this embodiment, the TAG preferably comprises a total of 5 double bonds in the fatty acid portions of the molecule. For instance, a triacylglycerol from TAG(46:5) to TAG(47:5) may be determined. In this embodiment, an increase in the level of the TAG in the sample from the subject compared to the reference value is indicative of an increased risk of unhealthy ageing in the subject. For instance, levels of TAG(46:5) or TAG(47:5) may be determined.

In another embodiment, the TAG comprises 48 to 54 carbon atoms in the fatty acid chains, and preferably 1 to 6 double bonds in the fatty acid portions. For instance, levels of a triacylglycerol from TAG(48:1) to TAG(54:6) may be determined. In this embodiment, a decrease in the level of the TAG in the sample from the subject compared to the reference value is indicative of an increased risk of unhealthy ageing in the subject. For instance, levels of TAG(48:6), TAG(52:2) or TAG(54:3) may be determined.

### Ether Phosphatidylcholines

In one embodiment, a ether phosphatidylcholine (PC-O) from PC-O(28:0) to PC-O(38:6) is determined (using the nomenclature (X:Y) as defined above). Thus a ether phosphatidylcholine (PC-O) from PC-O(28:0) to PC-O(38:6) refers to a PC-O comprising 28 to 38 carbon atoms in the fatty acid chain and 0 to 6 double bonds in the fatty acid chains. The present method may involve determining the level of one or more such individual species of PC-O.

In a preferred embodiment, the PC-O comprises 28 to 30 carbon atoms in the fatty acid portions of the molecule, and preferably no double bonds in the fatty acid portions. For instance, levels of PC-O(28:0) to PC-O(30:0) may be determined. In this embodiment, an increase in the level of the PC-O in the sample from the subject compared to the reference value is indicative of an increased risk of unhealthy ageing in the subject. For instance, levels of PC-O(28:0) or PC-O(30:0) may be determined.

In a preferred embodiment, the PC-O comprises 32 to 38 carbon atoms in the fatty acid portions of the molecule, and preferably 1 to 6 double bonds in the fatty acid portions. For instance, levels of PC-O(32:1) to PC-O(38:6) may be determined. In this embodiment, a decrease in the level of the PC-O in the sample from the subject compared to the reference value is indicative of an increased risk of unhealthy ageing in the subject. For instance, levels of PC-O(32:1), PC-O(34:1), PC-O(34:2), PC-O(36:3), PC-O(38:4), PC-O(38:5), or PC-O(38:6) may be determined.

### Sphingomyelins

In one embodiment, a sphingomyelin (SM) from SM(33:1) to SM(50:4) is determined (using the nomenclature (X:Y) as defined above). Thus a sphingomyelin (SM) from SM(33:1) to SM(50:4) refers to a SM comprising 33 to 50 carbon atoms in the fatty acid chain and 1 to 4 double bonds in the fatty acid chains. The present method may involve determining the level of one or more such individual species of SM.

In a preferred embodiment, the SM comprises 33 to 42 carbon atoms in the fatty acid portions of the molecule, and preferably 1 to 4 double bonds in the fatty acid portions. For instance, levels of SM(33:1) to SM(42:4) may be determined. In this embodiment, a decrease in the level of the SM in the sample from the subject compared to the reference value is indicative of an increased risk of unhealthy ageing in the subject. For instance, levels of SM(33:1), SM(34:1), SM(36:1), SM(36:2), SM(38:2), SM(41:2), SM(42:2), SM(42:3) or SM(42:4). may be determined.

In a preferred embodiment, the SM comprises 50 carbon atoms in the fatty acid portions of the molecule, and preferably 1 double bond in the fatty acid portions. For instance, levels of SM(50:1) may be determined. In this embodiment, an increase in the level of the SM in the sample from the subject compared to the reference value is indicative of an increased risk of unhealthy ageing in the subject.

### Phosphatidylcholines

In one embodiment, a phosphatidylcholine (PC) from PC(32:1) to PC(40:5) is determined (using the nomenclature (X:Y) as defined above). Thus a phosphatidylcholine (PC) from PC(32:1) to PC(40:5) refers to a PC comprising a total of 32 to 40 carbon atoms in the fatty acid chains and a total of 1 to 5 double bonds in the fatty acid chains. The present method may involve determining the level of one or more such individual species of PC.

In one embodiment, a decrease in the level of the PC in the sample from the subject compared to the reference value is indicative of an increased risk of unhealthy ageing in the subject. For instance, levels of PC(14:0-18:1) or PC(16:0-18:1) may be determined. The nomenclature (X1:Y1-X2:Y2) refers to the number of carbon atoms (X) and double bonds (Y) in the first (1) and second (2) fatty acid chain of the PC species. Thus PC(14:0-18:1) comprises 14 carbon atoms and no double bonds in a first fatty acid chain, and 18 carbon atoms and 1 double bond in a second fatty acid chain.

### Phosphatidylinositols

In one embodiment, a phosphatidylinositol (PI) from PI(36:1) to PI(38:3) is determined (using the nomenclature (X:Y) as defined above). Thus a phosphatidylinositol (PI) from PI(36:1) to PI(38:3) refers to a PI comprising a total of 36 to 38 carbon atoms in the fatty acid chains and a total of 1 to 3 double bonds in the fatty acid chains. The present method may involve determining the level of one or more such individual species of PI.

In one embodiment, a decrease in the level of the PI in the sample from the subject compared to the reference value is indicative of an increased risk of unhealthy ageing in the subject. For instance, levels of PI(18:1-16:0) or PI(20:3-18:0) may be determined. The nomenclature (X1:Y1-X2:Y2) refers to the number of carbon atoms (X) and double bonds (Y) in the first (1) and second (2) fatty acid chain of the PI species. Thus PI(18:1-16:0) comprises 18 carbon atoms and 1 double bond in a first fatty acid chain, and 16 carbon atoms and no double bonds in a second fatty acid chain.

### Phosphatidylethanolamines

In one embodiment, a phosphatidylethanolamine (PE) from PE(36:2) to PE(38:4) is determined (using the nomenclature (X:Y) as defined above). Thus a phosphatidylethanolamine (PE) from PE(36:2) to PE(38:4) refers to a PE comprising 36 to 38 carbon atoms in the fatty acid chains and 2 to 4 double bonds in the fatty acid chains. The present method may involve determining the level of one or more such individual species of PE.

In one embodiment, a decrease in the level of the PE in the sample from the subject compared to the reference value is indicative of an increased risk of unhealthy ageing in the subject.

### Combinations of Biomarkers

Whilst individual lipid biomarkers may have predictive value in the methods of the present invention, the quality and/or the predictive power of the methods is improved by combining values from multiple lipid biomarkers in the prediction of risk of unhealthy ageing.

Thus in general the method of the present invention may involve determining the level of at least two lipid biomarkers from those defined above, in particular at least one lipid biomarker from each of two or more lipid groups as defined above. In further embodiments, the method may comprise determining a level of any combination of two or more of the above lipid species in the sample. For instance, the method may comprise determining levels of 2, 3, 4, 5 or 10 or more lipid species as described above. The following combinations of lipid species are particularly preferred.

In one embodiment, the method comprises determining levels of a triacylglycerol from TAG(46:1) to TAG(54:6) and an ether phosphatidylcholine from PC-O(28:0) to PC-O(38:6).

In another embodiment, the method comprises determining levels of a triacylglycerol from TAG(46:1) to TAG(54:6), an ether phosphatidylcholine from PC-O(28:0) to PC-O(38:6) and a sphingomyelin (SM) from SM(33:1) to SM(50:4).

In another embodiment, the method comprises determining levels of a triacylglycerol from TAG(46:1) to TAG(54:6), an ether phosphatidylcholine from PC-O(28:0) to PC-O(38:6), a sphingomyelin (SM) from SM(33:1) to SM(50:4) and a phosphatidylcholine (PC) from PC(32:1) to PC(40:5).

In another embodiment, the method comprises determining levels of a triacylglycerol from TAG(46:1) to TAG(54:6), an ether phosphatidylcholine from PC-O(28:0) to PC-O(38:6), a sphingomyelin (SM) from SM(33:1) to SM(50:4), a phosphatidylcholine (PC) from PC(32:1) to PC(40:5) and a phosphatidylinositol (PI) from PI(36:1) to PI(38:3).

In another embodiment, the method comprises determining levels of a triacylglycerol from TAG(46:1) to TAG(54:6), an ether phosphatidylcholine from PC-O(28:0) to PC-O(38:6), a sphingomyelin (SM) from SM(33:1) to SM(50:4), a phosphatidylcholine (PC) from PC(32:1) to PC(40:5), a phosphatidylinositol (PI) from PI(36:1) to PI(38:3) and a phosphatidylethanolamine (PE) from PE(36:2) to PE(38:4).

### Comparison to Control

The present method further comprises a step of comparing the level of the individual lipid species in the test sample to one or more reference or control values. Typically a specific reference value for each individual lipid species determined in the method is used. The reference value may be a normal level of that lipid species, e.g. a level of the lipid in the same sample type (e.g. serum or plasma) in a normal subject. The reference value may, for example, be based on a mean or median level of the lipid species in a control population of subjects, e.g. 5, 10, 100, 1000 or more normal subjects (who may either be age- and/or gender-matched or unmatched to the test subject).

The extent of the difference between the subject's lipid biomarker levels and the corresponding reference values is also useful for characterizing the extent of the risk and thereby, determining which subjects would benefit most from certain interventions. Preferably the level of the lipid in the test sample is increased or decreased by at least 1%, 5%, at least 10%, at least 20%, at least 30%, or at least 50% compared to the reference value.

In some embodiments, the reference value is a value obtained previously from the same subject. This allows a direct comparison of the effects of a current lifestyle of the subject compared to a previous lifestyle on lipid biomarker levels and risk of unhealthy ageing, so that improvements can be directly assessed.

The reference value may be determined using corresponding methods to the determination of lipid levels in the test sample, e.g. using one or more samples taken from normal subjects. For instance, in some embodiments lipid levels in control samples may be determined in parallel assays to the test samples. Alternatively, in some embodiments reference values for the levels of individual lipid species in a particular sample type (e.g. serum or plasma) may already be available, for instance from published studies. Thus in some embodiments, the reference value may have been previously determined, or may be calculated or extrapolated, without having to perform a corresponding determination on a control sample with respect to each test sample obtained.

### Association of Lipid Levels to Risk of Unhealthy Ageing

In general, an increased or decreased level of any of the above lipid species in the test sample compared to the reference value may be indicative of an increased or decreased risk of unhealthy ageing in the subject, particularly an increased or decreased risk of developing age-related chronic inflammatory disease. The overall risk of unhealthy ageing in the subject may be assessed by determining a number of different lipid biomarkers as discussed above, and combining the results. For instance, subjects may be stratified into low, medium, high and/or very high risk groups according to the number of individual lipid species which are modulated relative to control and/or the degree to which they are elevated.

The advantage of assessing more than one biomarker is that the more biomarkers are evaluated the more reliable the diagnosis will become. If, e.g., more than 1, 2, 3, 4, 5, 6, or 7 biomarkers exhibit the elevations or decreases in levels as described above, this is indicative of highly increased risk of unhealthy ageing in the subject.

### Methods for Promoting Healthy Ageing

In one aspect, the present invention provides a method for promoting healthy ageing in a subject. In particular, the method may be used to reduce the risk of age-related chronic inflammatory conditions in the subject, or to improve longevity in the subject.

The method for promoting healthy ageing typically comprises a first step of determining a risk of unhealthy ageing in the subject by a method as described above. Following the determination of the risk of unhealthy ageing, an appropriate intervention strategy (e.g. a change in lifestyle and/or diet) may be selected for the subject, based on assessed risk level.

Typically if the subject shows a low level of risk of unhealthy ageing, no intervention may be necessary. For instance, if the subject's risk level is at or below a threshold level, no pharmaceutical or nutritional therapy may be required. The threshold level may correspond, for example, to a normal or mean level of risk in the general population.

Alternatively, if the subject shows an elevated risk of unhealthy ageing, the method may comprise a further step of modifying a lifestyle of the subject. The modification in lifestyle in the subject may be any change as described herein, e.g. a change in diet, more exercise, more sleep, less alcohol, less stress, less smoking, a different working and/or living environment.

Preferably the change is the use of at least one nutritional product that was previously not consumed or consumed in different amounts, e.g. a nutritional product that has an effect on healthy ageing and/or on avoiding ageing related chronic inflammatory disorders (including food products, drinks, pet food products, food supplements, nutraceuticals, food additives or nutritional formulas). In particularly preferred embodiments, the change in diet comprises an increased consumption of fish, fish oil, omega-3-polyunsaturated fatty acids, zinc, vitamin E and/or B vitamins.

Modifying a lifestyle of the subject also includes indicating a need for the subject to change his/her lifestyle, e.g. prescribing, promoting and/or proposing a lifestyle change as described above to the subject. For instance, the method may comprise a step of administering or providing at least one nutritional product as described above to the subject.

An advantage of the present invention is that a lifestyle modification can be selected which is effective in reducing levels of the specific lipid species associated with unhealthy ageing which are modulated in an individual subject. Typically, different lifestyle modifications (e.g. individual nutritional products) may have differing effects on the profiles of individual lipid species in individual subjects, due to various factors such as genetic variability and environment.

Thus in embodiments of the present invention, the lifestyle modification may be personalized to the subject, such that unhealthy ageing-risk associated lipid levels are monitored in conjunction with a specific program targeted to reducing those individual lipid species in the subject. For instance, the method may comprise a further step of (re-)determining lipid levels in the subject (i.e. after the initial lifestyle or diet-based intervention), in order to assess the effectiveness of the therapy in reducing the risk of unhealthy ageing. If the subject shows a reduction in risk of unhealthy ageing after the initial intervention phase, the intervention may be continued to maintain the risk at reduced levels.

However, if the subject fails to respond adequately to the initial intervention (e.g. shows no significant reduction in specific lipid levels and/or risk of unhealthy ageing), the subject may be switched to an alternative program, e.g. a different lifestyle modification, diet or nutritional agent. For example, if a subject responds poorly to an initial nutritional regime, an alternative nutritional product may be administered to the subject. This process may be repeated, including selecting different dosages of individual agents, until a reduction in unhealthy ageing risk-associated lipid levels is achieved. Typically, the subject may be maintained on a particular regime (e.g. a nutritional agent such as those defined above) for at least 1 week, 2 weeks, 1 month or 3 months before the determination of lipid levels is repeated. The method may be used to monitor the effects of lifestyle changes (such as changes in diet, exercise levels, smoking, alcohol consumption and so on) on unhealthy ageing-risk associated lipid levels, and to identify an combination of factors which is effective in reducing the risk of unhealthy ageing.

In a further aspect, the present invention provides a nutritional agent as defined above (e.g. selected from food products, drinks, pet food products, food, nutraceuticals, food additives or nutritional formulas), for use in promoting healthy ageing (or preventing or treating unhealthy ageing) in a subject, wherein a risk of unhealthy ageing in the subject has been determined by a method as described above and wherein the subject shows an increased risk of unhealthy ageing.

In a further aspect, the present invention provides use of a nutritional agent as defined above, for the manufacture of a medicament for promoting healthy ageing (or preventing or treating unhealthy ageing) in a subject, wherein a risk of unhealthy ageing in the subject has been determined by a method as described above and wherein the subject shows an increased risk of unhealthy ageing.

### Kits

In a further aspect, the present invention provides a kit for determining a risk of unhealthy ageing in a subject. The kit may, for example, comprise one or more reagents, standards and/or control samples for use in the methods described herein. For instance, in one embodiment the kit comprises one or more reference samples comprising predetermined levels of (i) a triacylglycerol (TAG) from TAG(46:1) to TAG(54:6); (ii) an ether phosphatidylcholine (PC-O) from PC-O(28:0) to PC-O(38:6); (iii) a sphingomyelin (SM) from SM(33:1) to SM(50:4); (iv) a phosphatidylcholine (PC) from PC(32:1) to PC(40:5); (v) a phosphatidylinositol (PI) from PI(36:1) to PI(38:3); (vi) a phosphatidylethanolamine (PE) from PE(36:2) to PE(38:4), and instructions for use of the kit for determining a risk of unhealthy ageing in a subject by comparing the predetermined levels in the reference sample to levels of lipids in a sample obtained from the subject. The kit may comprise control samples suitable for use with any combination of preferred lipid species defined above.

Those skilled in the art will understand that they can freely combine all features of the present invention described herein, without departing from the scope of the invention as disclosed. The invention will now be described by way of example only with respect to the following specific embodiments.

### Examples

Population aging has emerged as a major demographic trend worldwide due to improved health and longevity. This global aging phenomenon will have a major impact on health-care systems worldwide due to increased morbidity and greater needs for hospitalization/institutionalization. As the life expectancy of population increases worldwide, there is an increasing awareness of the importance of “healthy aging” and “quality of life”. Indeed, aging appears to be characterized by an increasing chronic, low grade inflammatory status indicated as inflamm-aging [1,2] and this condition is believed to be pathogenic contributing to frailty and degenerative disorders. Aging is a very complex process since several biochemical processes happen to the human organisms during the entire course of life, affecting all levels, from organ to cells, and leading to a wide variety of altered biochemical functions. However, this process is not completely understood. A large amount of data indicates that inflammation is strictly connected to oxidative stress. However, this process is not completely understood. A large amount of data indicates that inflammation is strictly connected to oxidative stress. Inflamm-aging is responsible for the majority of age-related diseases, such as cardiovascular disease (CVD), diabetes mellitus, Alzheimer disease (AD), and cancer [3-5] and for the most of deaths in elderly. Centenarians seem to be spared from inflamm-aging possessing a complex and peculiar balancing between pro-inflammatory and anti-inflammatory characteristics, resulting in a slower, more limited and balanced development of inflamm-aging, in comparison with old people, who are characterized by either faster or inadequately counteracted anti-inflammatory responses [6].

Systems-level omics technologies are emerging as a valuable approach to comprehensively investigate changes in metabolic regulations, and then linking these to the phenotypic outcome, thereby capturing the complexity and the multifactorial origin of aging [7-9]. In order to better elucidate molecular mechanisms that involve the disruption of lipid metabolic pathways, the field of lipidomics is also being rapidly emerging. Lipidomics can be achieved by a comprehensive measurement of the lipidome, i.e. the complete set of biological lipids, from a single analysis in a non-targeted profiling way (shotgun approach) [10]. In women, nineteen species comprising ether phosphocholines and sphingomyelins were found to associate with familial longevity and thereby identified as candidate longevity markers.

The longevity group consists of centenarians (average age 101 years, ±2), who is a well-accepted model of healthy human aging [1,2,11]. The control aging group consists of elderly individuals (average age 70 years±6). All subjects were recruited in Northern Italy. Our study confirms ether phosphocholine (PC-O) and sphingomyelin (SM) as markers of healthy aging, further strengthening the hypothesis that longevity is marked by better antioxidant ability and attained lipid mediated network able to maintain membrane composition and integrity within the increased inflamm-aging.

A MS/MS shot gun lipidomics approach was applied on serum samples from 15 centenarians, 37 elderly (Table 1). Multivariate data analysis was performed using Random Forests (RF™) (Breiman, L., Random Forests, Machine Learning, 2001, 45:5-32) on relative quantitative data from thirteen different lipid classes: triacylglycerol TG (n=30), sphingomyelin SM (n=25), lysophosphatidylcholine LPC (n=7), phosphatidylcholine PC (n=34), ether phosphatidylcholine PC-O (n=19), ceramides Cer (n=6), phosphatidylethanolamine PE (n=14), phosphatidylethanolamine based ether PE-O (n=9), lysophosphatidylethanolamine LPE (n=3), phosphatidylinositol PI (n=7), phosphatidic acid PA (n=1), diacylglycerol DAG (n=19).

Using the variable importance feature implemented in RF™, it was possible to determine the metabolic signature that better discriminates among elderly and centenarians. To assess the individual discriminant ability of each component of the signature paired t tests (2 tailed) was performed. Compared to elderly (Tables 3-4-5), relative concentration of sphingolipids increased in centenarians (Cer 42:2, SM 33:1, SM 34:1, SM 36:1, SM 36:2, SM 38:2, SM 41:2, SM 42:2, SM 42:3, SM 33:1, SM 42:4) glycerolphospholipids levels varies for selected species (increased in LPC 18:1, PC 14:0/18:1, PC 16:0/18:1, PC 16:0/18:2, PC 14:0/18:2, PC 16:0/18:3, PC 18:0/22:5; decreased in saturated PC-O 28:0, PC-O 30:0, increased in polyunsaturated PC-O 32:1, PC-O 34:1, PC-O 34:2, PC-O 36:3, PC-O 32:1, PC-O 38:4, PC-O 38:5, PC-O 38:6; increased in PE 16:0/20:4, PE 18:0/20:2, PE 18:0/20:3, PE 18:0/20:4; increase in PI 18:0/18:1, PI 18:1/16:0, PI 20:3/18:0; increased in SM 33:1, SM 34:1, SM 36:1, SM 36:2, SM 38:2, SM 41:2, SM 42:2, SM 42:3, SM 42:4, SM 50:), and glycerol lipids increased/decreased (decreased in TG 46:5, TG 47:5, DAG 26:0, DAG 26:1, increased in TG 48:6, TG 52:2, TG 54:3). The majority of centenarians are female individuals (Table 1), therefore performing gender separation leads to limited statistical power. However for qualitative indication we report values for females and males (Tables 4-5), displaying the overall trend is kept.

In the present work, in order to better assess changes in lipids profiling, we deployed a shot-gun lipidomics approach, able to quantify thirteen lipid family species. Here, we observed that centenarians display an overall increase in SM, which are important cellular messengers, with their low level associated to neurodegenerative diseases [12], atherosclerosis[13], and cardiovascular disease[14]. In our study, among the ten SM whose levels are higher in centenarians, three species are of particular interest; SM 41:2, SM 36:2, SM 34:1. SMs have previously been associated to familial longevity [15].

SM can be converted to ceramides by the enzymatic activities of sphingomyelinases (SMases). It was suggested that SMases activity increase with age [16], therefore increasing ceramide contents, with their accumulation negatively effecting pro-inflammatory pathologies [17,18]. In atherogenesis, for example, ceramide accumulation is linked to aggregation of LDL, increased ROS, and promotion of foam cell formations [19]. However our data reflect that among the six measured ceramide only one (Cer 42:2) increases, confirming previous finding on the lipidome signature of longevity and the notion that centenarians are somehow protected against the increasing inflammatory conditions.

Overall our increase in SM is in agreement with previous findings that certain cells have well adapted mechanism to cope against chronic oxidative stress by altering sphingomyelin metabolism, making changes to membrane composition [20]. This is also confirmed by overall increase in polyunsaturated ether PC (PC-O), plasmalogen species, able to prevent oxidation of lipoproteins and cardioprotective [21].

A large amount of data indicates that inflammation is strictly connected to oxidative stress. Reactive oxygen species (ROS) are continuously produced by cells as by-product of oxidative metabolism and are essential for several physiological functions, however an imbalance between the production of oxidants and protective antioxidant systems in favour of an excessive accumulation of ROS may cause cellular oxidative damage to nucleic acids and proteins in cells of several systems including the endocrine (Vitale et al., 2013) and the immune (Salvioli et al., 2013).

Changes in the phospholipids distribution influence membrane protein function, modifying the permeability of solutes across the membrane [22] through changes in the fluidity of the bi-layer. Measurement of the fatty acid composition of human erythrocyte membrane lipids has shown that centenarians have a reduced susceptibility to peroxidative membrane damage, while higher membrane fluidity compared with all the other age groups [23]. In particular, the increase in PE is interesting as it was previously postulated that highly polyunsaturated PE can carry pro-inflammatory molecules such as the arachidonic acid lipid network [24].

Another phospholipid, phosphatidylinositol (PI), possesses immunoregulatory capacities [25]. In our study we detect an increase in three PI species (PI 18:0/18:1, PI 18:1/16:0, PI 20:3/18:0) in centenarians. In animal tissues, phosphatidylinositol is the primary source of the arachidonic acid required for biosynthesis of eicosanoids, including prostaglandins, via the action of the enzyme phospholipase A2. We have previously displayed that centenarians possess a unique balanced of anti- and pro-inflammatory eicosanoids, therefore we believed that an increase in PE and PI mirrors these findings, displaying that centenarians possess an unique and effective modulation of the arachidonic acid metabolic cascade to counteract their inflammatory status.

Longevity is also characterized by decrease concentration of long chain triglycerides (TG 46:5, TG 47:5) and increase concentrations of very long TG chain with a high carbon number (TG 48:6, TG 52:2, TG 54:3). While usually highly unsaturated TG are target to peroxidation and the overall triglycerides family are seen as an adverse risk factor, recent investigations points to specific TG linked to adverse events where lipids of higher carbon number and double bond content were associated with decreased risk [26]. Our centenarians present an overall net balance among increase/decrease TG species compared to elderly individuals.

Lastly, we also noted that concentration of diacylglycerols decreases (DAG 26:0, DAG 26:1). DAG can result from the phosphatidic acid pathway, which represents the lipogenesis route in the synthesis of TAG and phospholipids. Most studies to date have clearly implicated DAGs derived from this pathway in activation of PKCE and hepatic insulin resistance. However intracellular DAGs can also be derived from TAG hydrolysis of lipid droplets, mediated by adipose triglyceride lipase (ATGL), and activation of phospholipase C, which will release DAGs from membrane lipids. Recent evidence support the hypothesis that increases in intracellular diacylglycerol content, due to an imbalance between fatty acid delivery and intracellular fatty acid oxidation and storage, leads to activation of new protein kinase C (PKC) isoforms that in turn inhibit insulin action in liver and skeletal muscle [27].

Overall the represented changes reflect that longevity is marked by better counteractive antioxidant capacity and a well-developed membrane lipid remodelling process able to maintain cell integrity.

### Experimental

### Subjects and Study Groups.

A total of 294 subjects belonging to two age groups were enrolled from four Italian cities (Bologna, Milan, Florence, Parma). The group of centenarians consisted of 98 subjects (mean age 100.7±2.1 yrs) born in Italy between the years 1900 and 1908. The elderly group includes 196 subjects (mean age 70±6 yrs). The study protocol was approved by the Ethical Committee of Sant'Orsola-Malpighi University Hospital (Bologna, Italy). Overnight fasting blood samples were obtained in the morning (between 7 and 8 a.m.). Serum was obtained after clotting and centrifugation at 760 g for 20 min at 4° C., and immediately frozen and stored at −80° C. After obtaining written informed consent, a standard questionnaire was administered by trained physicians and nursing staff to collect demographic and lifestyle data, anthropometric measurements, functional, cognitive and health status, clinical anamnesis.

### Clinical Chemistry.

Overnight fasting blood samples were obtained early in the morning. Serum total and HDL cholesterol, triglycerides, CRP, insulin resistance (HOMA-IR) were determined using standard hematology methods.

### Automated Sample Preparation for Shot-Gun Lipidomics

A 96 samples high throughput, fully automated liquid/liquid extraction method utilizing a Hamilton Microlabstar robot (Hamilton, Bonaduz, Switzerland) was developed in house for lipidomics extraction with minor modifications from previous methods[28]. Briefly, 5 μL of serum was used for delipidation. Lipid extraction was performed with 700 μL MTBE/MeOH (10/3) containing an internal standard mixture of 5 μM TAG 44:1, 0.5 μM DAG 24:0, 5 μM PC 28:0, 1 μM LPC 14:0, 1 μM PE 28:0, 0.5 μM LPE 14:0, 1 μM PS 28:0, 0.5 μM LPS 17:1, 1 μM PI 32:0, 0.5 μM LPI 17:1, 0.5 μM PA 28:0, 0.5 μM LPA 14:0, 1 μM PG 28:0, 0.5 μM LPG 14:0, 2 μM SM 35:1, 1 μM Cer 32:1. Samples were vortexed at 4° C. for 1 hour, followed by the addition of 150 μL water to induce phase separation. After centrifugation for 10 min at 5,000 g, 500 μL of the upper organic phase was transferred into a 96-deepwell-plate (Eppendorf, Hamburg, Germany), sealed with aluminum foil and stored at −20° C. until analysis. Prior to MS analysis 10 μL of total lipid extract were finally diluted with 90 μL of MS running buffer (isopropanol/methanol/chloroform 4:2:1 (v/v/v) containing 7.5 mM ammonium acetate).

### Identification and Quantification of Lipid Species in Plasma and Liver Extracts

Analysis was carried out on an LTQ Orbitrap Velos MS (Thermo Fisher Scientific, Reinach, Switzerland) system coupled to a Nanomate nanoinfusion ion source (Advion Bioscience Ltd, Harlow, Essex, UK). For each sample extract, two consecutive injections were realized for negative and positive ionization mode, respectively. Centroided high collisional dissociation (HCD) negative MS/MS were acquired in DDA mode. Each DDA cycle consisted of one MS survey spectra acquired at the target resolution Rm/z400 of 100,000, followed by the acquisition of 20 HCD FT MS/MS spectra at the resolution Rm/z400 of 30,000. One DDA experiment was completed in 25 min. Precursor ions were subjected to MS/MS if their m/z matched the masses of a pre-compiled inclusion list with the accuracy of 5 ppm. In positive ionization mode MS spectra were acquired at the target resolution Rm/z400 of 100,000, no further MS/MS experiments were performed. The lock mass option was enabled using LPA 17:0 (m/z 424.492; negative mode) and d18:1/17:0 Cer (m/z 551.528; positive mode) as reference peaks.

Lipid species were identified by LipidXplorer following the protocol of Herzog and co-workers. Data were then exported and further processed by an in-house developed software tool. The routine merged the data sets and generated Excel-output-files containing the normalized values (Internal standard to analyte ratio) and absolute concentrations by comparing the abundances of precursor ions of analyte and internal standard spiked prior to extraction.

### Chemicals and Lipid Standards

Ethanol, chloroform and iso-Propanol (HPLC grade) were purchased from Biosolve (Valkenswaard, the Netherlands). Methanol, water and Ammoniumacetate were obtained from Merck (Darmstadt, Germany). Synthetic lipid standards were purchased from Avanti Polar Lipids with purities higher than 99%. Stock-solutions of individual lipid compounds were prepared in methanol and stored at −20° C. Working solutions of the desired concentrations were prepared by dilution in isopropanol/methanol/chloroform 4:2:1 (v/v/v).

### Lipid Nomenclature

Lipids have been named according to Lipid Maps (http://www.lipidmaps.org) with the following abbreviations: PC, Phosphatidylcholine; PC-O, Phsophatidylcholine-ether; LPC, Lysophosphatidylcholine; PE, Phosphatidylethanolamine; PE-O, Phsophatidylethanolamine-ether; LPE, Lysophosphatidylethanolamine; PS, Phosphatidylserine; LPS, Lysophosphatidylserine; PI, Phosphatidylinositol; LPI, Lysophosphatidylinositol; PG, Phosphatidylglycerol; Cer, Ceramide; SM, Sphingomyelin; DAG, Diacylglycerol; TAG, Triacylglycerol, Phosphatidic acid; PA.

Individual lipid species were annotated as follows: [lipid class] [total number of carbon atoms]:[total number of double bonds]. For example, PC 34:4 reflects a phosphatidylcholine species comprising 34 carbon atoms and 4 double bonds.

### Multivariate Data Analysis.

Multivariate Data Analysis (MVA) was performed in several software environments. Thus, data import and pre-processing steps for both 1H NMR and targeted MS data were done using ‘in-house’ routines written in MATLAB (version 7.14.0, The Mathworks Inc., Natick, Mass., USA) and R (R Core Team (2012). R: A language and environment for statistical computing. R Foundation for Statistical Computing, Vienna, Austria. ISBN 3-900051-07-0, URL http://www.R-project.org/.). Targeted MS data was analyzed by Random Forests by using the package ‘randomForest’(A. Liaw and M. Wiener (2002). Classification and Regression by randomForest. R News 2(3), 18-22.) running in the R environment. Univariate significance tests for were also performed in R using the package ‘stats’. A level of significance of 0.05 or less was considered significant. MUFA to PUFA ratio were calculated by adding levels of all MUFA lipids (one double bonds in the acyl chains) and resulting value divided by sum of all PUFA lipids (two or more double bonds in the acyl chains).

### REFERENCE LIST

1. Sansoni P, Vescovini R, Fagnoni F, Biasini C, Zanni F et al. (2008) The immune system in extreme longevity. Exp Gerontol 43: 61-65.
2. Franceschi C, Capri M, Monti D, Giunta S, Olivieri F et al. (2007) Inflammaging and anti-inflammaging: a systemic perspective on aging and longevity emerged from studies in humans. Mech Ageing Dev 128: 92-105.
3. Franceschi C (2007) Inflammaging as a major characteristic of old people: can it be prevented or cured? Nutr Rev 65: S173-S176.
4. Burkle A, Caselli G, Franceschi C, Mariani E, Sansoni P et al. (2007) Pathophysiology of ageing, longevity and age related diseases. Immun Ageing 4: 4.
5. Franceschi C, Capri M, Monti D, Giunta S, Olivieri F et al. (2007) Inflammaging and anti-inflammaging: a systemic perspective on aging and longevity emerged from studies in humans. Mech Ageing Dev 128: 92-105.
6. Franceschi C, Bonafe M (2003) Centenarians as a model for healthy aging. Biochem Soc Trans 31: 457-461.
7. Nicholson J K, Holmes E, Wilson I D (2005) Gut microorganisms, mammalian metabolism and personalized health care. Nat Rev Microbiol 3: 431-438.
8. Li M, Wang B, Zhang M, Rantalainen M, Wang S et al. (2008) Symbiotic gut microbes modulate human metabolic phenotypes. Proc Natl Acad Sci USA 105: 2117-2122.
9. Martin F-P J, Wang Y, Sprenger N, Yap I K S, Rezzi S et al. (2008) Top-down Systems Biology Integration of Conditional Prebiotic Transgenomic Interactions in a Humanized Microbiome Mouse Model. Mol Syst Biol 4: 205.
10. Ejsing C S, Sampaio J L, Surendranath V, Duchoslav E, Ekroos K et al. (2009) Global analysis of the yeast lipidome by quantitative shotgun mass spectrometry. Proc Natl Acad Sci USA 106: 2136-2141.
11. Cevenini E, Invidia L, Lescai F, Salvioli S, Tieri P et al. (2008) Human models of aging and longevity. Expert Opin Biol Ther 8: 1393-1405.
12. Piccinini M, Scandroglio F, Prioni S, Buccinna B, Loberto N et al. (2010) Deregulated sphingolipid metabolism and membrane organization in neurodegenerative disorders. Mol Neurobiol 41: 314-340.
13. Nelson J C, Jiang X C, Tabas I, Tall A, Shea S (2006) Plasma sphingomyelin and subclinical atherosclerosis: findings from the multi-ethnic study of atherosclerosis. Am J Epidemiol 163: 903-912.
14. Holland W L, Summers S A (2008) Sphingolipids, insulin resistance, and metabolic disease: new insights from in vivo manipulation of sphingolipid metabolism. Endocr Rev 29: 381-402.
15. Gonzalez-Covarrubias V, Beekman M, Uh H W, Dane A, Troost J et al. (2013) Lipidomics of familial longevity. Aging Cell 12: 426-434.
16. Smith A R, Visioli F, Frei B, Hagen T M (2006) Age-related changes in endothelial nitric oxide synthase phosphorylation and nitric oxide dependent vasodilation: evidence for a novel mechanism involving sphingomyelinase and ceramide-activated phosphatase 2A. Aging Cell 5: 391-400.
17. Schmitz G, Grandl M (2007) Role of redox regulation and lipid rafts in macrophages during Ox-LDL-mediated foam cell formation. Antioxid Redox Signal 9: 1499-1518.
18. Grandl M, Bared S M, Liebisch G, Werner T, Barlage S et al. (2006) E-LDL and Ox-LDL differentially regulate ceramide and cholesterol raft microdomains in human Macrophages. Cytometry A 69: 189-191.
19. Bismuth J, Lin P, Yao Q, Chen C (2008) Ceramide: a common pathway for atherosclerosis? Atherosclerosis 196: 497-504.
20. Clement A B, Gamerdinger M, Tamboli I Y, Lutjohann D, Walter J et al. (2009) Adaptation of neuronal cells to chronic oxidative stress is associated with altered cholesterol and sphingolipid homeostasis and lysosomal function. J Neurochem 111: 669-682.
21. Wiesner P, Leidl K, Boettcher A, Schmitz G, Liebisch G (2009) Lipid profiling of FPLC-separated lipoprotein fractions by electrospray ionization tandem mass spectrometry. J Lipid Res 50: 574-585.
22. Farooqui A A, Ong W Y, Horrocks L A (2004) Biochemical aspects of neurodegeneration in human brain: involvement of neural membrane phospholipids and phospholipases A2. Neurochem Res 29: 1961-1977.
23. Rabini R A, Moretti N, Staffolani R, Salvolini E, Nanetti L et al. (2002) Reduced susceptibility to peroxidation of erythrocyte plasma membranes from centenarians. Exp Gerontol 37: 657-663.
24. Maskrey B H, Bermudez-Fajardo A, Morgan A H, Stewart-Jones E, Dioszeghy V et al. (2007) Activated platelets and monocytes generate four hydroxyphosphatidylethanolamines via lipoxygenase. J Biol Chem 282: 20151-20163.
25. van Dieren J M, Simons-Oosterhuis Y, Raatgeep H C, Lindenbergh-Kortleve D J, Lambers M E et al. (2011) Anti-inflammatory actions of phosphatidylinositol. Eur J Immunol 41: 1047-1057.
26. Rhee E P, Cheng S, Larson M G, Walford G A, Lewis G D et al. (2011) Lipid profiling identifies a triacylglycerol signature of insulin resistance and improves diabetes prediction in humans. J Clin Invest 121: 1402-1411.
27. Erion D M, Shulman G I (2010) Diacylglycerol-mediated insulin resistance. Nat Med 16: 400-402.
28. Matyash V, Liebisch G, Kurzchalia T V, Shevchenko A, Schwudke D (2008) Lipid extraction by methyl-tert-butyl ether for high-throughput lipidomics. J Lipid Res 49: 1137-1146.

All references described herein are incorporated by reference. Although the invention has been described by way of example, it should be appreciated that variations and modifications may be made without departing from the scope of the invention as defined in the claims. Furthermore, where known equivalents exist to specific features, such equivalents are incorporated as if specifically referred to in this specification. Further advantages and features of the present invention are apparent from the figures and non-limiting examples.

TABLE 1
Demographic, clinical characteristics of the
recruited aging cohort. Values are presented as mean (^{±}SD) with
the range in parentheses.

| Demographic | Centenarians | Elderly |
|---|---|---|
| Shot-gun | 3/12 | 16/21 |
| lipidomics | 100 ^{±} 1.3 | 69.7 ^{±} 16.1 |
| Gender, | (99-104) | (56-83) |
| male/female | | |
| Age, years | | |

TABLE 2
Clinical characteristics of the cohort as per shot-
gun lipidomics. Values are presented as mean (±SD) with the
range in parentheses. P value is as follow: *p < 0.05.,
**p < 0.01, ***p < 0.001.

| Clinical | Centenarians | | Elderly | |
|---|---|---|---|---|
| BMI, kg/m² | 22.5 ^{±} 3.8 | (17.8-29.2) | 27.2 ^{±} 1.5 | (16.1-49.7)** |
| HOMA index | 1.5 ^{±} 0.66 | (0.3-2.1) | 3.0 ^{±} 2.8 | (0.7-16.4)* |
| Cholesterol, mg/dl | 185.0 ^{±} 32.7 | (112-264) | 201.0 ^{±} 37.2 | (5-335)** |
| Triglycerides, mg/dl | 114.4 ^{±} 46.1 | (60-283) | 129.9 ^{±} 65.7 | (44-530)*** |
| HDL, mg/dl | 48.2 ^{±} 13.1 | (25-99) | 55.2 ⁺ 20.4 | (20-147)* |
| LDL, mg/dl | 105.6 ^{±} 35.1 | (75-165 | 118.7 ^{±} 45.7 | (23.8-199)* |
| CRP, mg/L | 3.97 ^{±} 4.9 | (0.28-19.9) | 3.24 ^{±} 3.9 | (0.11-19.1)* |
| A-SAA, μg/ml | 527 ^{±} 707 | (15.5-3821) | 142.9 ^{±} 187 | (0.01-1318)** |
| MMSE¹ | 24.2 ^{±} 4.2 | (15.2-23.2) | 27.16 ^{±} 1.53 | (23-29.7)*** |

| | | |
|---|---|---|
| Cardiovascular therapy, % | 33 | 67 |
| Irregular heart rhythm, % | 14 | 21 |
| Diabetes², % | 5 | 11 |

Legend:
BMI = body mass index,
HOMA = Homeostatic Model Assessment index,
HDL = high density lipoprotein,
LDL = low density lipoprotein,
CRP = C reactive protein,
A-SAA = Serum amyloid A (SAA) proteins.
¹MMSE = Cognitive function measure using the Mini-Mental State Examination (MMSE). The score used in the analysis was corrected by age and years of educations according to Magni et. al for old people. MMSE for elderly cognitive impairment was graded as severe (score 0-17), mild (score 18-23), or not present (score 24-30). MMSE for centenarians ≧20 absence of severe cognitive decline; <12 presence of severe cognitive decline according to Franceschi et al. 2000a.
²Diabetes mellitus: history of diabetes, fasting glucose plasma ≧126 mg/dl

TABLE 3
Lipid values are presented as mean (±SD) with the
range in parentheses. P value is as follow: *p < 0.05.,
**p < 0.01, ***p < 0.001. Table represent females and males
individuals.

| Lipid | Centenarians | Elderly |
|---|---|---|
| species [μM/l] | Mean ± SD | Mean ± SD |
| Cer 42:2 | 2.35 ± 0.76 | 0.42 ± 0.39*** |
| DAG 26:0 | 0.26 ± 0.66 | 3.41 ± 1.19*** |
| DAG 26:1 | 0.32 ± 0.87 | 2.68 ± 0.85*** |
| LPC 18:1 | 24.2 ± 3.53 | 16.8 ± 5.02*** |
| PC 14:0/18:1 | 23.4 ± 8.79 | 7.04 ± 4.83*** |
| PC 16.0/18.1 | 351 ± 61.3 | 168 ± 59.2*** |
| PC 16.0/18.2 | 599 ± 140 | 392 ± 114*** |
| PC 16.0/18.3 | 13.6 ± 4.6 | 3.68 ± 3.65*** |
| PC 18.0/22.5 | 12.3 ± 3.82 | 2.26 ± 2.56*** |
| PC-O 28:0 | 19.5 ± 3.02 | 47.6 ± 8.96*** |
| PC-O 30:0 | 34.5 ± 3.20 | 78.7 ± 13.3*** |
| PC-O 32:1 | 2.04 ± 1.65 | 0.11 ± 0.45*** |
| PC-O 34:1 | 8.02 ± 2.44 | 0.81 ± 1.50*** |
| PC-O 34:2 | 8.52 ± 3.16 | 2.01 ± 2.60*** |
| PC-O 36:3 | 4.11 ± 2.62 | 0.07 ± 0.42*** |
| PC-O 38:4 | 8.15 ± 2.94 | 1.22 ± 1.80*** |
| PC-O 38.5 | 22.1 ± 4.65 | 9.94 ± 5.02*** |
| PC-O 38.6 | 4.91 ± 3.37 | 0.37 ± 1.27*** |
| PE 16:0/20:4 | 1.77 ± 1.19 | 0.139 ± 0.50* |
| PE 18:0/20:2 | 2.02 ± 1.05 | 0.221 ± 0.42*** |
| PE 18:0/20:3 | 1.50 ± 0.81 | 0.08 ± 0.28*** |
| PE 18:0/20:4 | 7.84 ± 2.43 | 3.83 ± 2.25*** |
| PE 18:2/18:0 | 7.87 ± 1.92 | 3.32 ± 1.99*** |
| PI 18:0/18:1 | 2.37 ± 0.97 | 0.68 ± 0.65*** |
| PI 18:1/16:0 | 2.97 ± 1.17 | 0.80 ± 0.63*** |
| PI 20.3/18:0 | 5.55 ± 0.66 | 2.62 ± 1.20*** |
| SM 33:1 | 11.9 ± 2.43 | 6.09 ± 2.88*** |
| SM 34:1 | 150 ± 21.3 | 99.2 ± 23.7*** |
| SM 36:1 | 25.1 ± 5.04 | 16.5 ± 4.97*** |
| SM 36:2 | 10.8 ± 3.16 | 5.56 ± 3.42* |
| SM 38:2 | 5.54 ± 2.26 | 1.29 ± 1.23*** |
| SM 41:2 | 14.6 ± 3.67 | 7.92 ± 3.96* |
| SM 42:2 | 72.5 ± 12.2 | 44.2 ± 11.1*** |
| SM 42:3 | 35.8 ± 7.04 | 20.2 ± 7.04*** |
| SM 42:4 | 1.63 ± 1.17 | 0.05 ± 0.30* |
| SM 50:1 | 3.95 ± 0.86 | 7.30 ± 2.00*** |
| TAG 46:5 | 10.8 ± 3.58 | 18.4 ± 6.45** |
| TAG 47:5 | 3.167 ± 2.72 | 7.53 ± 2.57** |
| TAG 48:6 | 13.3 ± 3.09 | 7.38 ± 7.84* |
| TAG 52:2 | 109.9 ± 34.4 | 57.0 ± 27.3*** |
| TAG 54:3 | 32.7 ± 13.198 | 15.3 ± 9.63*** |

TABLE 4
Lipid values are presented as mean (±SD) with the
range in parentheses. P value is as follow: *p < 0.05.,
**p < 0.01, ***p < 0.001. Table represent females individuals.

| Lipid species [μM/l] | Centenarians | Elderly |
|---|---|---|
| Males | Mean ± SD | Mean ± SD |
| Cer 42:2 | 2.56 ± 0.17 | 0.41 ± 0.59*** |
| DAG 26:0 | 0.590 ± 0.83 | 3.29 ± 1.64*** |
| DAG 26:1 | 0.49 ± 0.69 | 2.61 ± 1.25*** |
| LPC 18:1 | 27.3 ± 3.59 | 18.1 ± 8.21*** |
| PC 14:0/18:1 | 23.45 ± 0.63 | 5.88 ± 4.28*** |
| PC 16.0/18.1 | 327.9 ± 58.1 | 165.1 ± 53.9*** |
| PC 16.0/18.2 | 625.5 ± 151.2 | 370.6 ± 131.1*** |
| PC 16.0/18.3 | 14.4 ± 3.12 | 3.32 ± 3.78*** |
| PC 18.0/22.5 | 12.0 ± 2.53 | 2.31 ± 3.21*** |
| PC-O 28:0 | 19.5 ± 1.34 | 45.6 ± 13.3*** |
| PC-O/30:0 | 35.6 ± 2.36 | 74.7 ± 20.8*** |
| PC-O 32:1 | 2.51 ± 1.83 | 0.31 ± 0.71*** |
| PC-O 34:1 | 9.23 ± 2.24 | 1.11 ± 2.21*** |
| PC-O 34:2 | 8.81 ± 2.25 | 2.95 ± 3.44*** |
| PC-O 36:3 | 4.35 ± 1.32 | 0.43 ± 1.19*** |
| PC-O 38:4 | 9.71 ± 4.34 | 2.00 ± 1.17*** |
| PC-O 38.5 | 23.6 ± 6.31 | 10.5 ± 5.08*** |
| PC-O 38.6 | 4.73 ± 3.55 | 0.94 ± 1.98* |
| PE 16:0/20:4 | 1.51 ± 1.33 | 0.06 ± 0.51* |
| PE 18:0/20:2 | 1.68 ± 0.57 | 0.28 ± 0.61* |
| PE 18:0/20:3 | 1.12 ± 0.02 | 0.15 ± 0.04* |
| PE 18:0/20:4 | 6.48 ± 1.15 | 2.93 ± 1.94** |
| PE 18:2/18:0 | 7.53 ± 1.58 | 2.82 ± 1.98* |
| PI 18:0/18:1 | 2.378 ± 0.973 | 0.688 ± 0.657* |
| PI 18:1/16:0 | 1.60 ± 0.56 | 0.72 ± 0.81* |
| PI 20.3/18:0 | 5.71 ± 0.09 | 2.51 ± 1.29* |
| SM 33:1 | 12.2 ± 2.46 | 5.61 ± 3.15* |
| SM 34:1 | 151 ± 30.5 | 94.8 ± 25.8** |
| SM 36:1 | 22.9 ± 4.81 | 15.4 ± 5.86* |
| SM 36:2 | 8.71 ± 2.19 | 4.52 ± 3.69* |
| SM 38:2 | 4.45 ± 1.62 | 1.05 ± 1.19* |
| SM 41:2 | 14.09 ± 3.94 | 7.39 ± 3.21** |
| SM 42:2 | 75.7 ± 15.8 | 44.1 ± 12.1*** |
| SM 42:3 | 35.2 ± 7.31 | 19.3 ± 7.31** |
| SM 42:4 | 1.22 ± 0.91 | 0.08 ± 0.31* |
| SM 50:1 | 4.29 ± 1.31 | 6.79 ± 2.15* |
| TAG 46:5 | 10.14 ± 3.58 | 17.0 ± 3.47** |
| TAG 47:5 | 2.86 ± 2.07 | 6.60 ± 2.84** |
| TAG 48:6 | 11.7 ± 1.04 | 6.73 ± 4.24** |
| TAG 52:2 | 85.7 ± 10.0 | 51.5 ± 20.6** |
| TAG 54:3 | 22.8 ± 1.27 | 14.4 ± 10.5* |

TABLE 5
Lipid values are presented as mean (±SD) with the
range in parentheses. P value is as follow: *p < 0.05.,
**p < 0.01, ***p < 0.001. Table represent females individuals.

| Lipid | | |
|---|---|---|
| species [μM/l] | Centenarians | Elderly |
| Females | Mean ± SD | Mean ± SD |
| Cer 42:2 | 2.11 ± 1.00 | 0.65 ± 0.55* |
| DAG 26:0 | 0.51 ± 1.23 | 3.08 ± 1.16** |
| DAG 26:1 | 0.53 ± 1.15 | 2.42 ± 0.81* |
| LPC 18:1 | 22.9 ± 6.29 | 18.1 ± 4.59* |
| PC 14:0/18:1 | 22.1 ± 8.91 | 8.84 ± 4.99* |
| PC 16.0/18.1 | 342 ± 75.6 | 189.3 ± 71.7*** |
| PC 16.0/18.2 | 557 ± 168 | 442 ± 114*** |
| PC 16.0/18.3 | 12.3 ± 5.83 | 5.23 ± 4.34*** |
| PC 18.0/22.5 | 11.4 ± 5.1 | 3.63 ± 2.61*** |
| PC-O 28:0 | 21.7 ± 8.07 | 44.8 ± 10.8*** |
| PC-O 30:0 | 37.3 ± 10.1 | 75.3 ± 15.1*** |
| PC-O 32:1 | 1.74 ± 1.53 | 0.07 ± 0.31* |
| PC-O 34:1 | 7.04 ± 3.04 | 1.41 ± 2.09*** |
| PC-O 34:2 | 7.72 ± 3.87 | 2.27 ± 2.81*** |
| PC-O 36:3 | 3.71 ± 2.86 | 0.55 ± 1.82*** |
| PC-O 38:4 | 7.08 ± 2.81 | 1.86 ± 3.01*** |
| PC-O 38.5 | 19.94 ± 6.99 | 11.3 ± 5.84*** |
| PC-O 38.6 | 4.55 ± 3.32 | 0.91 ± 1.09*** |
| PE 16:0/20:4 | 1.70 ± 1.16 | 0.22 ± 0.61* |
| PE 18:0/20:2 | 2.02 ± 1.05 | 0.22 ± 0.42** |
| PE 18:0/20:3 | 1.47 ± 0.81 | 0.23 ± 0.21* |
| PE 18:0/20:4 | 7.71 ± 2.71 | 4.96 ± 2.16** |
| PE 18:2/18:0 | 7.37 ± 2.61 | 4.28 ± 2.14* |
| PI 18:0/18:1 | 2.37 ± 1.12 | 0.93 ± 0.79** |
| PI 18:1/16:0 | 2.86 ± 1.34 | 1.15 ± 0.66* |
| PI 20.3/18:0 | 5.24 ± 1.11 | 3.17 ± 1.54* |
| SM 33:1 | 10.9 ± 3.96 | 7.41 ± 3.18* |
| SM 34:1 | 141 ± 30.6 | 108.8 ± 23.5** |
| SM 36:1 | 24.1 ± 7.09 | 19.2 ± 5.22* |
| SM 36:2 | 10.5 ± 4.26 | 7.55 ± 3.71* |
| SM 38:2 | 5.35 ± 2.67 | 2.28 ± 2.36* |
| SM 41:2 | 13.5 ± 5.22 | 9.81 ± 4.52* |
| SM 42:2 | 67.5 ± 16.9 | 46.6 ± 11.0* |
| SM 42:3 | 33.3 ± 10.1 | 22.4 ± 7.22* |
| SM 42:4 | 1.59 ± 1.21 | 0.08 ± 0.37* |
| SM 50:1 | 4.01 ± 0.87 | 7.19 ± 2.00* |
| TAG 46:5 | 11.4 ± 3.96 | 18.5 ± 8.61* |
| TAG 47:5 | 3.62 ± 2.90 | 7.39 ± 3.05* |
| TAG 48:6 | 12.6 ± 4.88 | 9.01 ± 7.81* |
| TAG 52:2 | 110.3 ± 38.6 | 66.5 ± 28.3*** |
| TAG 54:3 | 32.6 ± 15.1 | 18.5 ± 10.8*** |

1. A method for predicting a risk of unhealthy ageing in a subject, comprising:
(a) determining a level of two or more lipid biomarkers in a sample from the subject, wherein the biomarkers are selected from two or more of the following groups:
(i) a triacylglycerol (TAG) from TAG(46:1) to TAG(54:6);
(ii) an ether phosphatidylcholine (PC-O) from PC-O(28:0) to PC-O(38:6);
(iii) a sphingomyelin (SM) from SM(33:1) to SM(50:4);
(iv) a phosphatidylcholine (PC) from PC(32:1) to PC(40:5);
(v) a phosphatidylinositol (PI) from PI(36:1) to PI(38:3);
(vi) a phosphatidylethanolamine (PE) from PE(36:2) to PE(38:4); and
(b) comparing the levels of the biomarkers in the sample to reference values;
wherein the levels of the biomarkers in the sample compared to the reference values are indicative of the risk of unhealthy ageing in the subject.

2. A method according to claim 1, comprising determining a level of:
(i) a triacylglycerol (TAG) from TAG(46:1) to TAG(54:6); and
(ii) an ether phosphatidylcholine (PC-O) from PC-O(28:0) to PC-O(38:6) in the sample from the subject.

3. A method according to claim 2, further comprising determining a level of a sphingomyelin (SM) from SM(33:1) to SM(50:4) in the sample from the subject.

4. A method according to claim 2, further comprising determining a level of a phosphatidylethanolamine (PE) from PE(36:2) to PE(38:4) in the sample from the subject.

5. A method according to claim 2, further comprising determining a level of a phosphatidylinositol (PI) from PI(36:1) to PI(38:3) in the sample from the subject.

6. A method according to claim 2, further comprising determining a level of a phosphatidylcholine (PC) from PC(32:1) to PC(40:5) in the sample from the subject.

7. A method according to claim 1, wherein a level of a TAG from TAG(46:5) to TAG(47:5) is determined, and an increase in the level of the TAG in the sample from the subject compared to the reference value is indicative of an increased risk of unhealthy ageing in the subject.

8. A method according to claim 7, wherein the TAG is TAG(46:5) or TAG(47:5).

9. A method according to claim 1, wherein a level of a TAG from TAG(48:3) to TAG(54:6) is determined, and a decrease in the level of the TAG in the sample from the subject compared to the reference value is indicative of an increased risk of unhealthy ageing in the subject.

10. A method according to claim 9, wherein the TAG is TAG(48:6), TAG(52:2) or TAG(54:3).

11. A method according to claim 1, wherein a level of a PC-O from PC-O(28:0) to PC-O(30:0) is determined, and an increase in the level of the PC-O in the sample from the subject compared to the reference value is indicative of an increased risk of unhealthy ageing in the subject.

12. A method according to claim 11, wherein the PC-O is PC-O(28:0) or PC-O(30:0).

13. A method according to claim 1, wherein a level of a PC-O from PC-O(32:1) to PC-O(38:6) is determined, and a decrease in the level of the PC-O in the sample from the subject compared to the reference value is indicative of an increased risk of unhealthy ageing in the subject.

14. A method according to claim 13, wherein the PC-O is PC-O(32:1), PC-O(34:1), PC-O(34:2), PC-O(36:3), PC-O(38:4), PC-O(38:5) or PC-O(38:6).

15. A method according to claim 1, wherein a level of a SM from SM(33:1) to SM(42:4) is determined, and a decrease in the level of the SM in the sample from the subject compared to the reference value is indicative of an increased risk of unhealthy ageing in the subject.

16. A method according to claim 15, wherein the SM is SM(33:1), SM(34:1), SM(36:1), SM(36:2), SM(38:2), SM(41:2), SM(42:2), SM(42:3) or SM(42:4).

17. A method according to claim 1, wherein a level of SM(50:1) is determined, and an increase in the level of the SM in the sample from the subject compared to the reference value is indicative of an increased risk of unhealthy ageing in the subject.

18. A method according to claim 1, wherein a level of a PE from PE(36:2) to PE(38:4) is determined, and a decrease in the level of the PE in the sample from the subject compared to the reference value is indicative of an increased risk of unhealthy ageing in the subject.

19. A method according to claim 1, wherein a level of a PI from PI(36:1) to PI(38:3) is determined, and a decrease in the level of the PI in the sample from the subject compared to the reference value is indicative of an increased risk of unhealthy ageing in the subject.

20. A method according to claim 19, wherein the PI is PI(18:1-16:0) or PI(20:3-18:0).

21. A method according to claim 1, wherein a level of a PC from PC(32:1) to PC(40:5) is determined, and a decrease in the level of the PC in the sample from the subject compared to the reference value is indicative of an increased risk of unhealthy ageing in the subject.

22. A method according to claim 21, wherein the PC is PC(14:0-18:1) or PC(16:0-18:1).

23. A method according to claim 1, wherein the sample comprises serum or plasma obtained from the subject.

24. A method according to claim 1, wherein the reference value is based on a mean level of the biomarker in a control population of subjects.

25. A method according to claim 1, wherein the levels of the biomarkers are determined by mass spectrometry.

26. A method according to claim 1, wherein the levels of the biomarkers in the sample compared to the reference values are indicative of the risk of developing chronic age-related inflammatory disease in the subject.

27. A method according to claim 1, wherein the levels of the biomarkers in the sample compared to the reference values are indicative of longevity of the subject.

28. A method for promoting healthy ageing in a subject, comprising:
(a) performing the method according to claim 1; and
(b) modifying a lifestyle of the subject if the subject has levels of the biomarkers which are indicative of an increased risk of unhealthy ageing.

29. A method according to claim 28, wherein the modification in lifestyle in the subject comprises a change in diet.

30. A method according to claim 29, wherein the change in diet comprises administering at least one nutritional product to the subject that reduces the risk of the development of chronic age-related inflammatory disease in the subject.

31. A method according to claim 29, wherein the change in diet comprises increased consumption of fish, fish oil, omega-3 polyunsaturated fatty acids, zinc, vitamin E and/or B vitamins.

32. A method according to claim 28, further comprising repeating step (a) after modifying the lifestyle of the subject.

33. A method for predicting a risk of unhealthy ageing in a subject, comprising:
(a) determining a level of a triacylglycerol (TAG) from TAG(46:1) to TAG(54:6) in a sample from the subject; and
(b) comparing the levels of the TAG in the sample to a reference value;
wherein the levels of the TAG in the sample compared to the reference value is indicative of the risk of unhealthy ageing in the subject.
